(19) United States
(12) Patent Application Publication
(10) Pub. No.: US 2018/0179479 A1
(43) Pub. Date: Jun. 28, 2018

(54) COMPRESSED YEAST FOR DIRECT INOCULATION OF A FRUIT OR VEGETABLE SUBSTRATE

(71) Applicant: Chr. Hansen A/S, Hoersholm (DK)

(72) Inventors: Kristine BJERRE, Virum (DK); Jan Hendrik SWIEGERS, Fredensborg (DK); Mansour BADAKI, Vanloese (DK); Katja Sander JENSEN, Hvidovre (DK)

(73) Assignee: Chr. Hansen A/S, Hoersholm (DK)

(21) Appl. No.: 15/578,915

(22) PCT Filed: Jun. 3, 2016
(86) PCT No.: PCT/EP2016/062705

§ 371 Date: Dec. 1, 2017

(30) Foreign Application Priority Data
Jun. 4, 2015 (EP) 15170659.5

(51) Int. Cl. C12G 1/02; C12N 1/18

(52) U.S. Cl. CPC C12G 1/02; C12G 2200/05; C12N 1/18

(57) ABSTRACT

The present invention relates to a novel form of compressed yeast for direct inoculation in the fermentation of a fruit or vegetable substrate, e.g. for the fermentation of beverages, such as wine or beer. Especially, the present invention relates to compressed yeast with a dry matter content of between 35% and 90% (w/w), preferably in a frozen form, a method for producing a fermented beverage by direct inoculation of a fruit or vegetable substrate with the compressed yeast and a container comprising the compressed yeast.

### FIELD OF THE INVENTION

The present invention relates to a novel form of compressed yeast for direct inoculation in the fermentation of a fruit or vegetable substrate, e.g. for the fermentation of beverages, such as wine or beer. Especially, the present invention relates to compressed yeast with a dry matter content of between 35% and 90% (w/w), preferably in a frozen form, a method for producing a fermented beverage by direct inoculation of a fruit or vegetable substrate with the compressed yeast and a container comprising the compressed yeast.

### BACKGROUND OF THE INVENTION

In the wine industry the yeast used is normally active dried yeast (ADY) with a dry-matter content of more than 90% (w/w) which needs time-consuming re-hydration and re-activation at specific temperature before inoculation into the wine.

The production of ADY involves several steps where the yeast is first produced in a fermentor, concentrated, filtrated and lastly includes fluid bed drying of the compressed yeast.

However, this process means the product needs to go through several steps (re-hydration, re-activation and acclimatization) before it can be used for inoculation.

Because of the drying of the product, the yeast cells are dehydrated and therefore needs to be re-hydrated and then re-activated in suitable media in order to be metabolic active before application to the substrate (e.g. grape juice as in the case of winemaking). This is a very delicate process for the yeast cells and requires a significant amount of time and attention since factors such as temperature, timing and activation media are important to ensure the survival of the cells. Thus, the rehydration of ADY usually demands a large number of skilled man-hours at a commercial winery during the winemaking process.

During the drying process of active dried yeast production, the cellular membranes of the yeast cells lose their permeability barrier function (Roger Boulton et al, 1996, page 124). Therefore, to re-establish this function, it is important to re-hydrate the membranes by adding the yeast in water at 40° C. for 20 minutes. Therefore, the yeast re-hydration process typically involves a 20-30 minute rehydration in un-chlorinated water or a water/grape juice mix (2:1) at a temperature of between 35-38′C, followed by the addition of grape juice of the same volume (50:50 juice/water blend) which is kept for another 20-30 minutes before adding it to wine. It is important that the grape juice does not contain any SO₂, which could kill the yeast cells during the sensitive process of rehydration (O'Kennedy, 2008). In addition, the rehydration mix must be cooled down with juice after 20 min in water, 5° C. at a time. Failure to cool down from the rehydration temperature after 30 minutes can also result in significant cell death (O'Kennedy, 2008). Furthermore, care should be taken to use uncontaminated grape juice for the rehydration protocol as rehydration with contaminated grape juice will result in contamination of all wine fermentation inoculated using the rehydration mixture. Different manufacturers propose variations on this protocol but the critical step is that dehydrated cells need to be exposed to water or a water/juice mixture at specific temperatures, under sanitary conditions and for specific times in order to hydrate properly, thereby avoiding cell death and consequent in-activity. It is not recommended to add the active dried yeast directly to juice since the high sugar concentration, SO₂ and other compounds in the grape juice do not allow for optimal rehydration of the yeast. For this reason, none of the wine yeast manufacturers propose the direct inoculation of active dried yeast to grape must.

Active-dried yeast (ADY) loses activity when not optimally rehydrated (Soubeyrand et al. 2006). The incorrect rehydration of ADY can also lead to stuck alcoholic fermentation (O'Kennedy, 2008), i.e. the yeast is not fermenting all of the sugar present in the substrate resulting in a beverage that is too sweet.

Another way to add yeast to a substrate is to use frozen yeast that can be added directly to the substrate (WO2011/134952). In this method the product is frozen cream yeast with a dry matter content below 28% (w/w) that allows for direct inoculation and high survival during direct inoculation. The concentrate is frozen at −50° C. The disadvantage of this method is that it is crucial that the product is frozen at −50° C., distributed by cold chain, and stored at −50° C. Furthermore, the harsh freezing conditions of the concentrate is damaging to the yeast cells affecting their viability. As a result, additives, which stabilize the yeast cells during and after freezing and/or drying, are often added to the liquid concentrate prior to freezing.

There is an increasing demand from consumers for so-called “clean label” food products, where the number of additives added to the products is limited. The food industry strives to follow the country-specific regulations and recommendations of the food authorities and intergovernmental organizations, such as OIV (International Organisation of Vine and Wine; http://www.oiv.int), EBC (European Brewery Convention) and ASBC (American Society for Brewing Chemists). Furthermore, the use of additives dilutes the concentrate resulting in a lower concentration of yeast cells (colony forming units (CFU) per volume of concentrate) in the final compressed yeast formulation.

### SUMMARY OF THE INVENTION

An object of the present invention is the provision of an improved formulation of yeast for fermentation of beverages by direct inoculation of a fruit or vegetable substrate which allows for a higher viability of the yeast in the absence of added additives, such as emulsifiers, oil, water-activity modifying agents and agents added to improve the stability of the yeast cells during freezing, drying and/or cold storage. In addition to the use in fermentation of beverages, it is contemplated that the yeast formulation according to the present invention is well suited for ethanol fermentation by direct inoculation of a carbohydrate-rich liquid substrate.

The present inventors have surprisingly found that compressed yeast, such as wine and brewer's yeast, with a dry matter content of between 35% to 90% (w/w) shows high survival when being frozen despite the relatively high moisture content and in the absence of agents added to improve the stability of the yeast cells during freezing, drying and/or cold storage. In addition, the compressed yeast is ideal for direct inoculation of fruit or vegetable substrates resulting in close to 100% survivability and the compressed yeast can be stored at 4° C. for several months under sanitary conditions. e.g. vacuum packed to avoid contamination, or stored for extended periods of time frozen.

### DETAILED DESCRIPTION OF THE INVENTION

FIG. 1 shows the different steps for down-stream processes for production of ADY and inoculation of a substrate with ADY (top) as well as the production of compressed yeast according to the present invention (bottom).

As can be seen in FIG. 1 (bottom) both the production of compressed yeast according to the present invention as well as the inoculation with compressed yeast according to the present invention is significantly shortened resulting in reduced costs of production.

The present invention in a first aspect relates to the provision of a compressed yeast for direct inoculation of a fruit or vegetable substrate with a dry matter content of between 35% and 90% (w/w). In some embodiments, the dry matter content is between 30% and 45%, such as between 30% and 40% or between 35% and 45%.

In another embodiment, the dry matter content of the compressed yeast is between 45% and 75% (w/w).

The dry matter content of the sample is measured by heating at 105° C.+/−5° C. in order to evaporate water content. The sample is measured before and after drying and the below calculations are carried out to get dry-matter content expressed as % (w/w):

The dry matter (W_{dm}) content expressed as a percentage of mass or grams per kilogram is calculated using the following equations:

$${w}_{dm} = \frac{{m}_{c} - {m}_{a}}{{m}_{b} - {m}_{a}} \times f$$

where:

W_{dm} is the dry matter of the sample, in percentages or grams per kilogram;

mₐ is the mass of the empty dish or crucible in grams;

m_{b} is the mass of the dish or crucible containing the sample in grams;

m_{c} is the mass of the dish or crucible containing the sample in grams after complete dehydration and removal of all water;

f is a conversion factor, f=100 for expression of results as a percentage and factor f=1000 for expression in grams per kilogram.

Values should be rounded to the nearest 0.1% (w/w) or alternatively to the nearest 1 g/kg.

In a preferred embodiment the compressed yeast according to the invention does not comprise any added additives.

In a preferred embodiment, the viability of the compressed yeast is at least 20% after freezing as calculated based on the concentration of CFU (colony forming units) of the compressed yeast before freezing and the concentration of CFU of the compressed yeast after freezing. Preferably, the viability of the yeast is at least 25%, such as at least 30%, such as at least 35%, such as at least 40%, such as at least 45%, such as at least 50%, such as at least 55%, such as at least 60%, at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99%. Most preferably, the viability is 100%.

In a preferred embodiment the viability of the compressed yeast is at least 60% after direct inoculation of a fruit or vegetable substrate as calculated based on the concentration of CFU (colony forming units) of the compressed yeast and the concentration of CFU of the inoculated material. Preferably, the viability of the yeast is at least 65%, such as at least 70%, such as at least 75%, such as at least 80%, such as at least 85%, such as at least 90%, such as at least 95%.

In another preferred embodiment the viability of the compressed yeast is at least 80% after storage for five months at a temperature of −20° C. followed by direct inoculation of a fruit or vegetable substrate as calculated based on the concentration of CFU (colony forming units) of the compressed yeast before storage and the concentration of CFU of the inoculated material. Preferably, the viability of the yeast is at least 85%, such as at least 90%, such as at least 95%.

In a preferred embodiment, the compressed yeast is frozen at a temperature below 0° C. Preferably, the compressed yeast is frozen at a temperature significantly below 0° C., such as at −5° C., such as at −20° C., such as at −50° C.

In a preferred embodiment, the frozen compressed yeast according to the present invention is frozen in the absence of any additives added to the liquid yeast concentrate in order to stabilize the yeast cells during and after freezing.

In a preferred embodiment, the viability of the frozen compressed yeast is at least 20% after freezing as calculated based on the concentration of CFU (colony forming units) of the compressed yeast before freezing and the concentration of CFU of the compressed yeast after freezing. Preferably, the viability of the yeast is at least 25%, at least 30%, such as at least 35%, such as at least 40%, such as at least 45%, such as at least 50%, such as at least 55%, such as at least 60%, at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99%. Most preferably, the viability is 100%.

The determination of the viability of yeast can be carried out by any suitable method known to the skilled person. In a preferred embodiment the determination of CFU/g and CFU/ml cell counts is performed as set out by OIV in chapter II of the International Oenological Codex, 2013 Issue.

In a preferred embodiment the viability of the frozen compressed yeast is at least 60% after direct inoculation of a fruit or vegetable substrate as calculated based on the concentration of CFU (colony forming units) of the compressed yeast and the concentration of CFU of the inoculated material. Preferably, the viability of the yeast is at least 65%, such as at least 70%, such as at least 75%, such as at least 80%, such as at least 85%, such as at least 90%, such as at least 95%.

In another preferred embodiment the viability of the frozen compressed yeast is at least 80% after storage for five months at a temperature of −20° C. followed by direct inoculation of a fruit or vegetable substrate as calculated based on the concentration of CFU (colony forming units) of the compressed yeast before freezing and storage and the concentration of CFU of the inoculated material. Preferably, the viability of the yeast is at least 85%, such as at least 90%, such as at least 95%.

The compressed yeast according to the present invention is preferably present in a concentrated form.

In a preferred embodiment the compressed yeast contains at least 10⁹ CFU/g of yeast, such as at least 5×10⁹ CFU/g of yeast, such as at least 10¹⁰ CFU/g of yeast, such as at least 5×10¹⁰ CFU/g of yeast, such as at least 10¹¹ CFU/g of yeast, such as at least 5×10¹¹ CFU/g of yeast, such as at least 10¹² CFU/g of yeast.

In a preferred embodiment the compressed yeast is selected from the genera of the group consisting of Saccharomyces, Kluyveromyces, Lachancea, Torulaspora, Brettanomyces, Pichia, Metschnikowla, Candida, Hanseniaspora, Saccharomycodes, Zygosaccharomyces, Cryptococcus, Issatchenkia, Schizosaccharomyces, Wickerhamomyces and Debaryomyces.

In a preferred embodiment the compressed yeast is a Saccharomyces yeast, preferably a Saccharomyces cerevisiae yeast

In another preferred embodiment the yeast is a wine yeast or a brewer's yeast, preferably the wine yeast or brewer's yeast is a yeast selected from the group consisting of Saccharomyces. Kluyveromyces, Lachancea, Torulaspora, Brettanomyces, Pichia and Metschnikowia yeast.

The invention further provides a method for producing a fermented beverage comprising the steps:

a) providing a fruit or vegetable substrate for production of the beverage;

b) directly inoculating the substrate with the compressed yeast according to the invention; and

c) fermenting the substrate with the compressed yeast to obtain the fermented beverage. As known to the skilled person directly inoculating means that the compressed yeast is added directly to the fruit or vegetable substrate without any re-hydration or re-activating steps.

The fermentation may be carried out under aerobic or anaerobic conditions or it may be carried out under a sequence of aerobic and anaerobic conditions.

In a preferred embodiment the fermented beverage is selected from the group consisting of wine, beer, cider, sake and soft-drinks. Preferably, the fermented beverage is wine.

In another preferred embodiment the fruit or vegetable substrate for production of the beverage is a fruit or vegetable juice. Preferably, the fruit juice is grape juice and the fermented beverage is wine.

It is contemplated that the compressed yeast according to the present invention will be equally suitable for use in a similar method of direct inoculation of an aqueous fruit or vegetable substrate for ethanol fermentation under anaerobic conditions. Suitable fruit or vegetable substrates are carbohydrate-rich substrates including but not limited to aqueous solutions based on corn syrup, cane sugar/molasses.

Another aspect of the present invention is related to a container comprising the compressed yeast according to the invention. In a preferred embodiment, the container is selected from the group consisting of a carton or a sealed plastic container.

### Definitions

The term “cream yeast” herein refers to liquid yeast with a dry matter content of below 28% (w/w) conventionally produced by propagation of yeast in a fermentor followed by concentration by centrifugation.

The term “compressed yeast” refers herein to a yeast with a dry matter content of between 35% and 90% (w/w) conventionally produced by propagation of yeast in a fermentor followed by concentration, filtration, extrusion and optionally partial drying on a drier, such as a fluid bed drier. In some embodiments, the dry matter content is between 30% and 45%, such as between 30% and 40% or between 35% and 45%.

Thus, the term “partially dried compressed yeast” refers herein to a yeast with a dry matter content of between 45% to 90% (w/w) produced by propagation of yeast in a fermentor followed by concentration, filtration, extrusion and partial drying on a drier, such as a fluid bed drier.

The term “active dried yeast” or “ADY” refers herein to yeast with a dry matter content of more than 90% (w/w) conventionally produced by propagation of yeast in a fermentor followed by concentration, filtration, extrusion and drying on a fluid bed drier.

The term “vegetable” refers herein to any plant. Preferably, the term “vegetable” herein refers to edible plants or edible plant parts.

The term “fruit” refers herein to the edible part of a plant developed from a flower. Fruit may include any accessory tissues of the edible part, such as the skin, peel or pod.

The term “yeast enhancer” herein refers to supplementary nutrients, such as vitamins, nitrogen, phosphate or minerals, added prior to or during propagation of the yeast.

The term “additives” herein refers to food-grade agents which are added to the yeast concentrate at any time point during production of the yeast formulation after propagation of the yeast, e.g. to assist in the extrusion and cutting of the yeast concentrate, e.g. emulsifiers, including, but not limited to, glycerol, glucose, sucrose and trehalose, and oil, to improve the stability of the yeast cells during freezing and/or cold storage, to change the melting point of the frozen yeast formulation, e.g. water-activity modifying agents, etc.

The term “added” when referring to additives herein refers to that the additives are introduced into the yeast concentrate during production of the compressed yeast in an amount efficient to give the desired effect.

### BRIEF DESCRIPTION OF THE FIGURES

FIG. 1 depicts the protocol for preparing ADY and for inoculation of ADY (top) as well as the protocol for preparing compressed yeast according to the present application for direct inoculation for the preparation of fermented beverages (bottom).

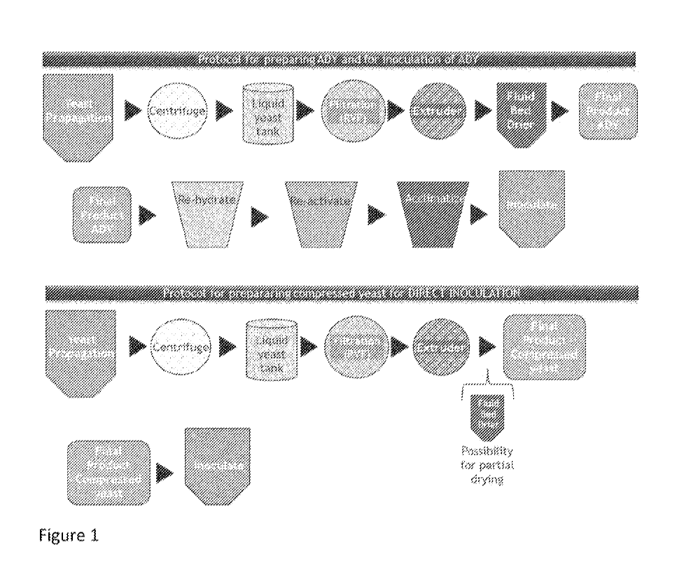

FIG. 2 shows the survival of Saccharomyces cerevisrae yeast either after keeping the concentrate at 4° C. or freezing the concentrate at −20° C., at −50° C. in the freezer or in liquid nitrogen and storing the yeast for 1 month at the indicated temperature.

FIG. 3 shows the survival of Saccharomyces cerevisiae yeast either after keeping the concentrate at 4° C. or freezing the concentrate at −20° C., at −50° C. in the freezer or in liquid nitrogen and storing the yeast for 1 month at the indicated temperature followed by direct inoculation in grape must.

FIG. 4 shows total survival of Saccharomyces cerevisiae yeast including both survival after treatment (FIG. 3) and survival in direct inoculation (FIG. 2).

FIG. 5 shows the survival of Saccharomyces cerevisiae yeast formulated as compressed yeast according to the present invention or as ADY before treatment and after storage for different time periods at the indicated temperatures.

FIG. 6 shows a comparison between the survival of yeast cells of the Saccharomyces cerevisiae ADY yeast and the Saccharomyces cerevisiae frozen compressed yeast according to the present invention after direct inoculation and standard inoculation for ADY yeast (“reactivation” of yeast), respectively.

### EXAMPLES

### Example 1

During the production of a conventional Saccharomyces cerevisiae strain, samples were taken out at three different stages during production; 1) as liquid (cream) yeast after centrifugation; 2) as compressed yeast (fresh yeast) with a dry matter content of 40% alter extrusion, and 3) as active dry yeast (ADY) with a dry matter content of 92% after fluid bed drying:

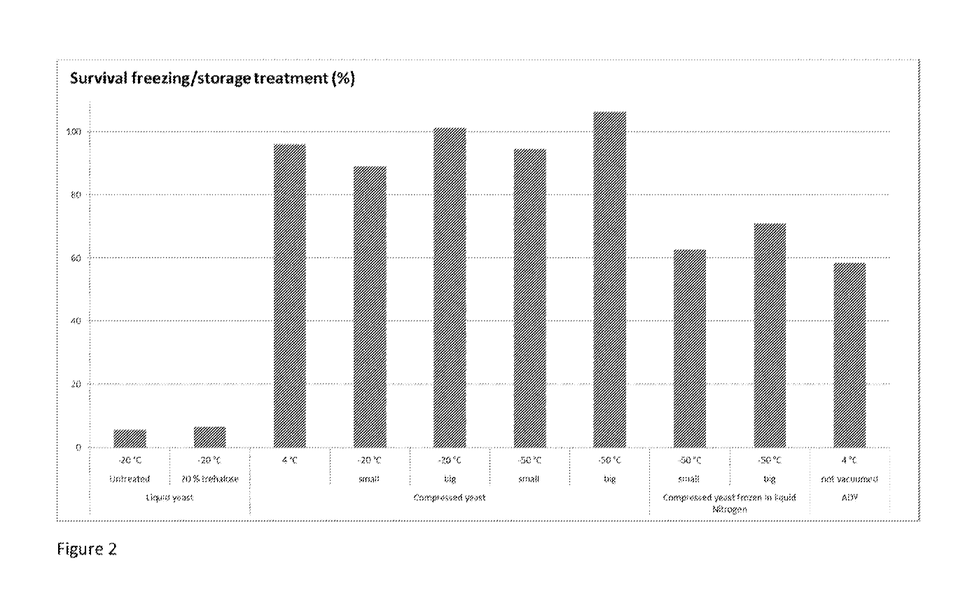

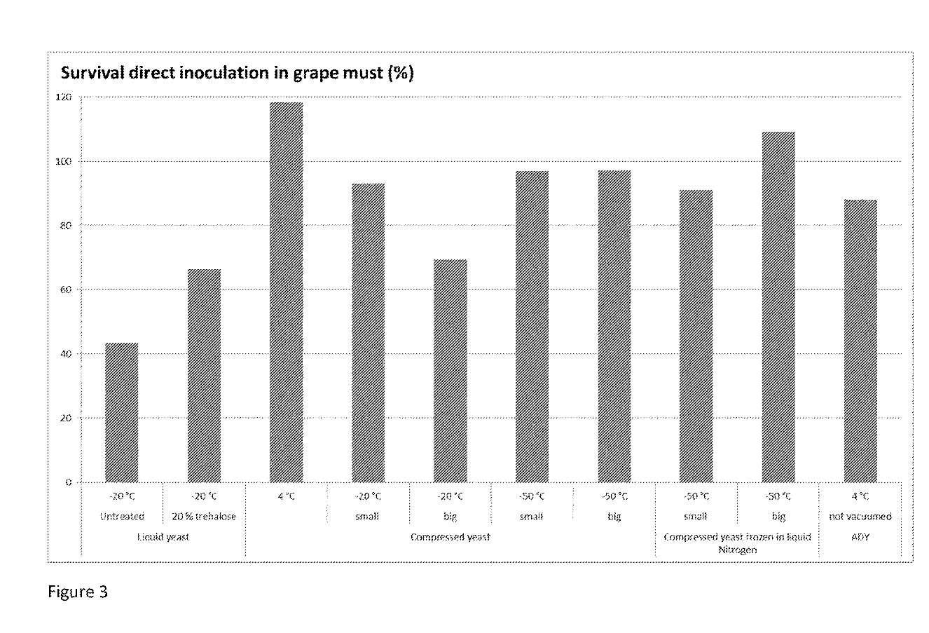

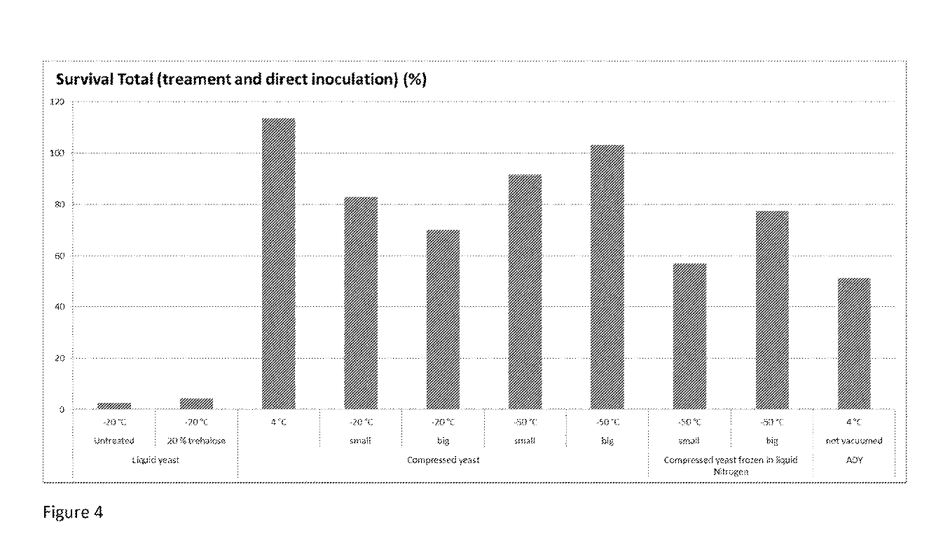

These samples were analysed according to cell count, survival after either keeping the concentrate at 4° C. or freezing the concentrate at −20° C., at −50° C. in the freezer or in liquid nitrogen and storing for 1 month at the indicated temperature (FIG. 2), survival after either keeping the concentrate at 4° C. or freezing the concentrate at −20° C., at −50° C. in the freezer or in liquid nitrogen and storing for 1 month at the indicated temperature followed by direct inoculation in grape must (FIG. 3), and total survival in direct inoculation in grape must after either keeping the concentrate at 4° C. or freezing the concentrate at −20° C. at −50° C. in the freezer or in liquid nitrogen and storing for 1 month at the indicated temperature (FIG. 4).

### Survival of Treatment and Over Time

The liquid yeast was frozen with 200 ml sample in a −20° C. freezer as untreated or with 20% trehalose. Cell counts were measured before and then again one month after freezing and followed over time.

The compressed yeast was frozen in different sizes and at different temperatures in order to verify the effect of the freezing rate on the survival of yeast cells. The sizes of the samples were 200 ml bottles (big) and 50 ml falcon tubes (small). The temperatures of the freezers were −20° C. and −50° C. respectively.

Also the compressed yeast was frozen in liquid nitrogen as pellets (1-2 ml long (small) and as 5 g clumps (big)).

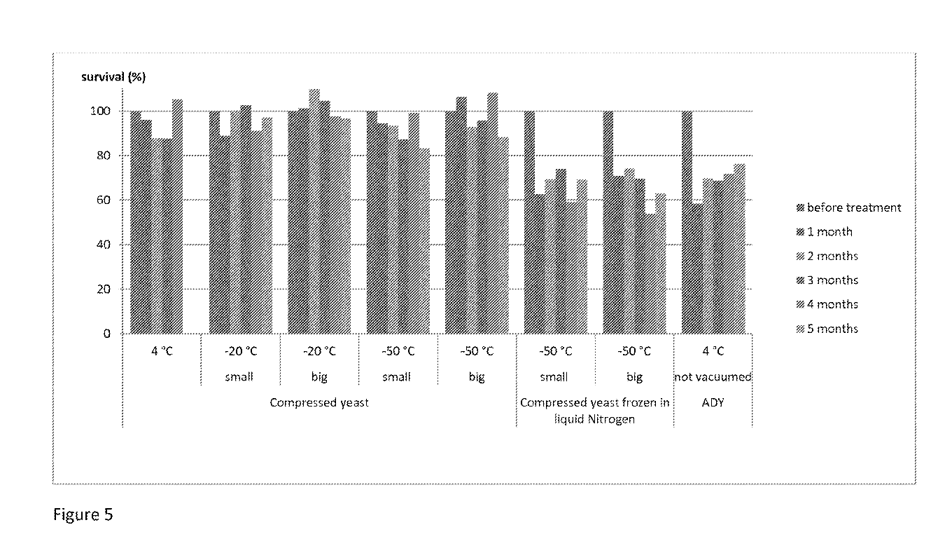

For determination of yeast survival (in percentages), cell counts were measured before freezing and again one month after freezing (FIG. 2) and followed over time (FIG. 5).

### Survival in Direct Inoculation

All samples were also used for direct inoculation after 1 month of storage in order to see the survival when the yeast cells were exposed to water and high osmotic pressure in the grape juice. 1.0 g of samples was added to 200 mL Riesling juice (Table 1). The samples were dissolved by gentle stirring and after approximately 10-15 min. the dilution series was performed and for determination of yeast survival (in percentages), CFU/g was calculated after direct inoculation (FIG. 3).

TABLE 1
Parameters of grape juice used for direct
inoculation experiments after 1 month

| Grape juice | Sugar (g/l) | Malic acid (g/l) | TSO₂ (mg/l) |
|---|---|---|---|
| Riesling (Germany) | 180 | 6.6 | 0 |

### Comparison of Survival by Direct Inoculation and “Reactivation” of the Yeast

Survival of inoculation by use of two different methods was compared. In both methods the inoculation volume was 0.2 g/l. For direct inoculation, the sample was added directly to the Chardonnay juice (Table 2). For standard inoculation, the sample was first rehydrated 1:10 in unchlorinated water for approximately half an hour. Un-sulphured grape juice was then added to the water in the ratio (1:3) and the suspension was then left to activate for approximately another 20 minutes. The final activated suspension was then added to the juice to reach a final inoculation level of 0.2 g/l. All inoculations were performed in duplicates.

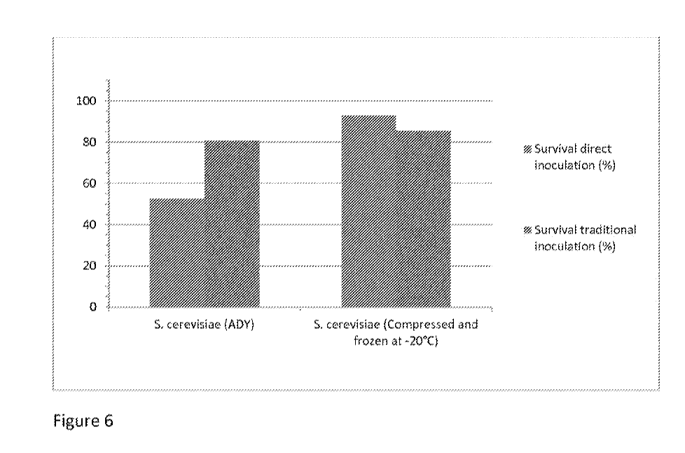

CFU/g of the products were calculated before inoculation and after inoculation and the survival was then calculated as percentages (FIG. 6).

TABLE 2
Parameters of grape juice used for direct
inoculation experiments after 8 months

| Grape juice | Sugar (g/l) | Malic acid (g/l) | TSO₂ (mg/l) |
|---|---|---|---|
| Chardonnay (Germany) | 180 | 6.7 | 0 |

### Determination of Colony Forming Units (CFU)

For determination of CFU/g and CFU/ml cell counts from samples taken from either inoculated grape juice or yeast dissolved in peptone water were performed by pour-plating on YGC media (prepared as set out by OIV in appendix VI of the International Oenological Codex, 2013 Issue). 1 ml of the sample from the dilution series (peptone water) was added to the plate and on top of that the liquid YGC agar (45° C.) was poured over the sample and mixed. After setting, the plates were incubated at 30° C. for 2-3 days. Determinations of CFU were in all cases performed in triplicates.

### Conclusion:

The results show that the survival of the yeast in direct inoculation (FIG. 2) is very high for especially compressed yeast stored at 4° C. and compressed yeast frozen at −50° C. or in liquid nitrogen.

The survival of the yeast after different treatments and storage of the samples for 1 month at this temperature shows high variations (FIG. 3). Surprisingly, the compressed yeast with high water content shows close to 100% survival when frozen at −20° C. and −50° C. When the compressed yeast is frozen in liquid nitrogen the survival (>60%) is also surprisingly high. The liquid yeast shows very low survival during freezing and the ADY also loose a high percentage of CFU when stored for 1 month at 4° C.

The overall survival shows that the compressed yeast, even when frozen, shows the highest survival (FIG. 4). The survival of the compressed yeast frozen at −50° C. and used in direct inoculation is close to 100%.

Stability of the different formats is measured over 5 months and results are shown in (FIG. 5). The cell counts of the compressed yeast samples are very stable even though the temperature has not been constant as the samples have been taken out several times.

The compressed yeast stored at 4° C. was only followed for 4 months as the sample was not packed in a proper sanitary way to be kept for a longer period of time.

The active dry yeast (ADY) products show lower survival when directly inoculated compared to when inoculated after rehydration and reactivation (FIG. 6). When used for direct inoculation the ADY shows an average survival of 53% and when inoculated after rehydration and reactivation the survival is 81% on average. For the frozen compressed yeast the survival in direct inoculation is 93% and survival when inoculated according to the “standard” procedure is 86% which is also higher than the average of the ADY products.

### REFERENCES

Roger Boulton, Vernon Singleton, Linda Bisson and Ralph Kunkee. 1996. Principles and Practices of Winemaking. pp. 124.
Karien O'Kennedy. (2008). How to avoid stuck fermentations, The Australian & New Zealand Grapegrower & Winemaker. November, Issue 538, 103-105.
Virginie Soubeyrand, Anne Jullen and Jean-Marie Sablayrolles (2006) Rehydration Protocols for Active Dry Wine Yeasts and the Search for Early Indicators of Yeast Activity American Journal of Enolology and Viticulture. 57(4)474-480.

1-12. (canceled)

13. A method for producing a fermented beverage, comprising:
(a) providing a fruit or vegetable substrate for production of the beverage;
(b) adding a frozen compressed yeast with a dry matter content of between 35% and 90% (w/w) to the fruit or vegetable substrate without any re-hydration or re-activating steps; and
(c) fermenting the fruit or vegetable substrate with the compressed yeast to obtain the fermented beverage.

14. The method according to claim 13, wherein the dry matter content of the frozen compressed yeast is between 45% and 75% (w/w).

15. The method according to claim 13, wherein the compressed yeast does not comprise any added additives.

16. The method according to claim 13, wherein the compressed yeast is frozen in the absence of any added additives added to the liquid yeast concentrate in order to stabilize the yeast cells during and after freezing.

17. The method according to claim 13, wherein the viability of the frozen yeast is at least 20% based on the concentration of CFU (colony forming units) of the compressed yeast before freezing and the concentration of CFU of the compressed yeast after freezing.

18. The method according to claim 13, wherein the viability of the yeast is at least 60% after step (b) as calculated based on the concentration of CFU (colony forming units) of the compressed yeast and the concentration of CFU of the material resulting from step (b).

19. The method according to claim 13, wherein the viability of the yeast is at least 80% after storage for five months at a temperature of −20° C. followed by step (b) as calculated based on the concentration of CFU (colony forming units) of the compressed yeast before freezing and the concentration of CFU of the material resulting from step (b).

20. The method according to claim 13, wherein the yeast is selected from Saccharomyces, Kluyveromyces, Lachancea, Torulaspora, Brettanomyces, Pichia, Metschnikowia, Candida, Hanseniaspora, Saccharomycodes, Zygosaccharomyces, Cryptococcus, Issatchenkia, Schizosaccharomyces, Wickerhamomyces and Debaryomyces.

21. The method according to claim 20, wherein the yeast is a Saccharomyces yeast.

22. The method according to claim 13, wherein the fermented beverage is selected from wine, beer, cider, sake and soft drinks.

23. The method according to claim 13, wherein the substrate is a fruit juice.

24. The method according to claim 23, wherein the fruit juice is grape juice and the fermented beverage is wine.
